(19) United States
(12) Patent Application Publication
(10) Pub. No.: US 2005/0042131 A1
(43) Pub. Date: Feb. 24, 2005

(54) Blood oxygenator with spacers

(76) Inventor: Mark J. Gartner, Pittsburgh, PA (US)

Correspondence Address: BLYNN L. SHIDELER;THE BLK LAW GROUP, 3500 BROKKTREE ROAD, SUITE 200, WEXFORD, PA, 15090, US

(21) Appl. No.: 10/919,080

(22) Filed: Aug. 16, 2004

Related U.S. Application Data
(60) Provisional application No. 60/496,599, filed on Aug. 20, 2003.

(51) Int. Cl. A61M 1/14

(52) U.S. Cl. 422/45

(57) ABSTRACT

An apparatus and method related to mass transfer and fluid pump devices. The apparatus generally includes a housing that defines an interior cavity, a diffuser positioned in the interior cavity of the housing, the diffuser comprising porous elements arranged in discrete layers, a diffusion area that is defined by a pre-determined distance between the discrete layers of porous elements. A fluid is accelerated through each diffuser layer and is then de-accelerated in the diffusion area.

### CROSS-REFERENCE TO RELATED APPLICATIONS

This application claims the benefit of U.S. Provisional Patent Application 60/496,599, filed Aug. 20, 2003, and entitled “Blood Oxygenator With Spacers”.

### BACKGROUND OF THE INVENTION

1. Field of the Invention

The present invention generally relates to fluid pumps and, more particularly, to a combination mass transfer/pump apparatus and method.

2. Brief Description of the Prior Art

Blood oxygenation pumps are generally shown in U.S. Pat. No. 6,348,175 to Borovetz et al.; U.S. Pat. No. 6,217,826 to Reeder et al.; U.S. Pat. No. 6,106,776 to Borovetz et al.; and U.S. Pat. No. 5,80,370 to Maloney, Jr. et al. Each of these patents is herein incorporated by reference in its entirety. Commercially, blood oxygenators are available from COBE under the OPTIMA mark, MEDTRONIC under the AFFINITY mark, and JOSTA under the QUADROX mark. All of these blood oxygenators are also incorporated by reference in their entirety.

The patents and devices incorporated above generally disclose pumps that include a permeable membrane/porous element/hollow fiber disk enclosed within a housing. In the patents listed above, the porous element rotates within the housing, and a fluid such as blood is pumped through the housing. The fluid in the housing physically contacts the fibers, which facilitates mass transfer between the fluid adjacent to an exterior surface of the diffuser and a fluid or fluids adjacent an interior surface of the diffuser. In the commercially available devices incorporated above, the permeable membrane or rolled sheet of porous fibers is stationary.

A particularly important application of this technology is blood oxygenation/de-carbonation. In active fluid transfer systems, blood is drawn into the housing via the rotating porous element and blood plasma comes into contact with one or more fibers. Because the fibers are gas permeable, carbon dioxide can diffuse from a red blood cell, through the blood plasma, through the fibers, and then be carried away through an exhaust lumen that is fluidly connected to corresponding fibers. Similarly, oxygen supplied by oxygen lumens can diffuse through the fibers, through the blood plasma, and to oxygen deprived blood cells.

In blood/gas transfer operations that use a rotary or active porous element, a common problem is the activation or destruction of blood elements caused by contact with the porous element. A balance must be obtained between disruption of the blood boundary layer surrounding the gas permeable diffuser fibers and inducing over-excessive shear. Although shear is minimized in the referenced prior art, improvements are still needed. A second known problem of rotary or static porous elements is the occlusion of micro-pores defined by the hollow fibers. Occlusion in blood pumping applications can be caused by blood plasma, as well as clotted blood.

Therefore, a need exists for a mass transfer pump that can reduce physical damage to blood cells and reduce occlusion of the fiber micro-pores.

### SUMMARY OF THE INVENTION

The present invention helps to reduce shear and increase available surface area of the porous elements/hollow fiber disks.

A fluid pump according to a preferred embodiment of the present invention generally includes a housing that defines an interior cavity, a diffuser positioned in the interior cavity of the housing, the diffuser including porous elements such as hollow fibers arranged in discrete layers, and a diffusion area that is defined by a pre-determined distance between the discrete layers of porous elements. The pre-selected distance is approximately equal to an outer diameter of the porous elements, such as 100-500 microns, with 300 microns being preferred.

The diffuser can be active or passive. An active or passive diffuser can generally include porous elements arranged as a sheet and then rolled into a cylinder. The pre-selected distance defines a space between adjacent discrete layers of the roll. A spacer or spacers can be used to help maintain the pre-selected distance between adjacent discrete layers of the roll. An active or rotary diffuser can also generally include two or more substantially parallel and discrete layers of porous elements, either arranged in a disk-like or cylinder-like configuration. A double lumen shaft may be attached to the two or more substantially parallel and discrete layers, wherein the double lumen shaft defines a gas inlet pathway and a gas outlet path. As noted above, a spacer or spacers may be used to maintain the pre-selected distance between the two of the two or more substantially parallel and discrete layers of porous elements. An optional nose cone may also be positioned on an exterior surface of one of the two or more substantially parallel and discrete layers of porous elements.

In an active or passive type of mass transfer device or pump, the housing generally defines a venous blood inlet and an arterial blood outlet. Moreover, the porous elements may be coated with sub-micron silicone to help prevent occlusion of the porous elements.

A method to improve fluid flow according to the present invention generally includes the steps of introducing a fluid into a diffuser comprising porous elements arranged in discrete layers; creating a diffusion area between the discrete layers of porous elements; accelerating the fluid through a first layer of porous elements; and de-accelerating the fluid in the diffusion area. In multiple layered diffusers, the fluid is repeatedly accelerated and de-accelerated through successive layers of porous elements and diffusing areas. The step of creating a diffusion area may further include the steps of determining on outer diameter of a hollow fiber in the porous element and making the diffusion area approximately equal to the outer diameter of the hollow fiber.

### BRIEF DESCRIPTION OF THE DRAWING

FIG. 1 is a cross-sectional side view of a rotary type pump according to the present invention; and

FIG. 2 is a cross-sectional end view of a static porous element fluid pump according to the present invention.

### DETAILED DESCRIPTION OF THE INVENTION

This application incorporates U.S. Provisional Patent Application No. 60/496,599, filed Aug. 20, 2003, and entitled “Blood Oxygenator With Spacers” by reference in its entirety.

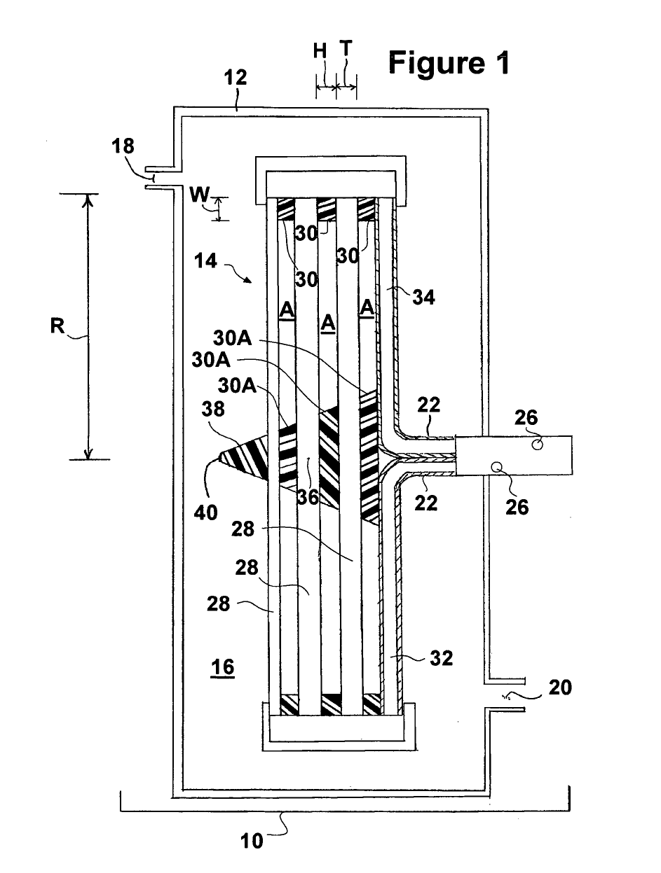

FIG. 1 shows an active fluid pump 10 according to a preferred embodiment of the present invention. The fluid pump 10 generally includes a housing 12 and a diffuser 14 that is rotatable with respect to the housing 12. The housing 12 generally defines an interior cavity 16, a venous blood inlet 18, and an arterial blood outlet 20.

The diffuser 14 generally includes a double lumen shaft 22 that defines a gas supply inlet 24 and a gas exhaust outlet 26, in addition to two or more stacked hollow fiber or porous element disks 28. One or more shims or spacers 30 are positioned between each disk 28. The disks 28 are preferably made from a plurality of non-wettable hollow fibers, or other suitable type of porous material. The hollow fibers each have an outer diameter and are bundled together to form the disks. The double lumen shaft 22 defines a gas inlet pathway 32 and a gas outlet pathway 34. The gas inlet pathway 32 is in fluid communication with the gas supply inlet 24 and the gas outlet pathway 34 is in fluid communication with the gas exhaust outlet 26.

The shims or spacers 30, preferably made from polycarbonate or other suitable material, may be positioned between the stacked hollow fiber disks 28, preferably at an outer periphery of each disk 28. In the preferred embodiment, the disks 28 each have a radius R of approximately two inches and a thickness T of approximately 0.5 inches. The shims 30 shown in FIG. 1 have a variable spacing height H of approximately 0.015 inches and a variable width W of approximately 0.2 inches. However, the height H of the shims 30, which in turn helps to define a diffusion area A between the disks 28, is preferably selected to match an outer diameter of a hollow fiber or porous element incorporated into the disks 28. For example, a range of 100-500 microns is contemplated, with a 300 micron height being preferred. The shims 30 are removable, and are preferably removed once the hollow fiber disks 28 are set with a setting material.

Tapered shims or spacers 30A are also preferably positioned at or about a center portion 36 of each disk 28, wherein the spacers 30A define a progressive taper from the double lumen shaft end (generally shown as 22) of the diffuser 14. A nose cone 38 may be used to complete the taper and define an apex 40 of the diffuser 14. The footprint of the first appearing tapered spacer 30A is approximately 0.4 inches in diameter for a two inch radial disk. The variable spacing height of each spacer 30A is approximately 0.015 inches in FIG. 1, but the height H can be adjusted to equal the outer diameter of a hollow fiber or other porous element that is incorporated into one of the disks 28. The tapered nose cone 38 has a height of approximately 0.5 inches. The spacer 30, 30A and disk 28 dimensions may also vary according the particular application.

The maximization of mass (or heat) exchange in the present active mixing mass exchange system for blood oxygenation/decarbonation system relies on exploiting the relative motion between the fibers in the diffuser and the blood. As the blood progresses through each rotating disc, the blood begins to accelerate in the direction of disc rotation due to entrainment of the blood by the fibers and the viscous transfer of momentum. As this entrainment occurs, the relative velocity between the fiber and the blood decreases causing mass (or heat) exchange to similarly decrease.

Consideration of the physics governing this process suggests that if the blood had an opportunity to decelerate between successive layers of fiber via a diffusing space, that these important relative velocities could be maximized during subsequent re-entry into the next fiber layer. This deceleration is demonstrated by noting the law of continuity:
V_{fiber}A_{fiber}=V_{no fiber}A_{no fiber}
Since the cross section area of the diffusing space is greater than the layer containing fiber and, since this area increases, the velocity of the blood in the layer must decrease. This maximizes the relative velocities when the blood enters the next layer containing fiber.

This effect may also have a positive benefit with respect to red cell hemolysis. It has been demonstrated that red cell damage is a function of both the magnitude of shear stress exposure and the time of this exposure. Hence, a red cell can tolerate a large shear for a short period of time, or a small shear for a long period of time, without substantial damage.

It has been found that the diffusing space acts as a “flow diffuser”, allowing the magnitude of the shear stress to relax for a period of time. This effect has been verified by observing that prototypes with spacers between the layers demonstrate significantly lower hemolysis than those without these spacers. In general, a nose cone with spacers works better than just the spacers alone. Moreover, the spacers alone work better than just the nosecone.

To help prevent clogging of the fibers, the fibers may be coated with sub-micron siloxane or other suitable material. It has been found that the sub-micron siloxane significantly prevents clogging of fiber pores by blood plasma and also helps prevent clotted blood cells from attaching to or being slightly intruding into the fiber pores.

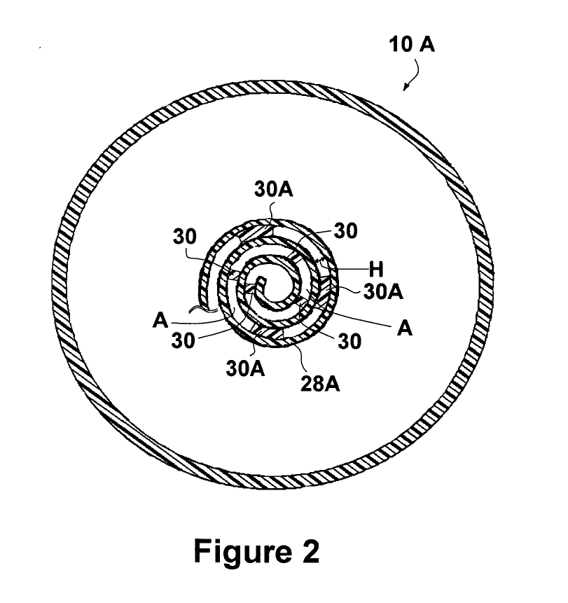

As noted above, the present invention is preferably used in fluid pumps and mass exchange devices, such as mass exchange devices having rotatable hollow fiber elements. However, the present invention is not limited to rotating diffusers. The spacers and cone described above may also be used in any hollow fiber membrane oxygenators, such as static type fluid pumps. Unlike the fluid pump 10 discussed above, each of the fluid pumps 10A (FIG. 2) generally include hollow fiber layers 28A, each comprising a plurality of bundled fibers. The layers are static and do not rotate with respect to the outer housing 12A.

As noted above, it has been observed that good results are achieved when the spacing A between layers 28A is approximately equal to the diameter of one of the fibers in one of the layers 28A. For example, a 300 micron gap or space between adjacent layers 28A is preferred when a porous element or hollow fiber contained in one of the layers 28A has outer diameter of approximately 300 microns. The space may be maintained by spacers 30 or 30A, which can be modified to define a partial cylindrical cross-section.

While specific embodiments of the invention have been described in detail, it will be appreciated by those skilled in the art that various modifications and alternatives to those details could be developed in light of the overall teachings of the disclosure. The presently preferred embodiments described herein are meant to be illustrative only and not limiting as to the scope of the invention which is to be given the full breadth of the appended claims and any and all equivalents thereof.

1. A fluid pump comprising:
a housing that defines an interior cavity;
a diffuser positioned in the interior cavity of the housing, the diffuser comprising porous elements arranged in discrete layers; and
a diffusion area that is defined by a pre-determined distance between the discrete layers of porous elements.

2. The fluid pump as claimed in claim 1, wherein the pre-selected distance is approximately equal to an outer diameter of the porous elements.

3. The fluid pump as claimed in claim 1, wherein the pre-selected distance is approximately 300 microns.

4. The fluid pump as claimed in claim 1, wherein the diffuser comprises a roll of porous elements and the pre-selected distance defines a space between adjacent discrete layers of the roll.

5. The fluid pump as claimed in claim 4, further comprises a removable spacer that maintains the pre-selected distance between adjacent discrete layers of the roll.

6. The fluid pump as claimed in claim 1, wherein the diffuser comprises two or more substantially parallel and discrete layers of porous elements.

7. The fluid pump as claimed in claim 6, further comprising a double lumen shaft that is attached to the two or more substantially parallel and discrete layers, wherein the double lumen shaft defines a gas inlet pathway and a gas outlet path.

8. The fluid pump as claimed in claim 6, further comprising a removable spacer that maintains the pre-selected distance between two of the two or more substantially parallel and discrete layers of porous elements.

9. The fluid pump as claimed in claim 6, further comprising a nose cone positioned on an exterior surface of one of the two or more substantially parallel and discrete layers of porous elements.

10. The fluid pump as claimed in claim 1, wherein the housing further defines a venous blood inlet and an arterial blood outlet.

11. The fluid pump as claimed in claim 1, wherein the diffuser comprises a gas supply inlet and a gas exhaust outlet.

12. The fluid pump as claimed in claim 1, wherein the porous elements are coated with sub-micron silicone-based compound.

13. A method to improve fluid flow comprising the steps of:
a) introducing a fluid into a diffuser comprising porous elements arranged in discrete layers;
b) creating a diffusion area between the discrete layers of porous elements;
c) accelerating the fluid through a first layer of porous elements; and
d) de-accelerating the fluid in the diffusion area.

14. The method as claimed in claim 13 wherein the step of creating a diffusion area further comprises the steps of determining on outer diameter of a hollow fiber in the porous element and making the diffusion area approximately equal to the outer diameter of the hollow fiber.
